(19) United States
(12) Patent Application Publication
(10) Pub. No.: US 2010/0009434 A1
(43) Pub. Date: Jan. 14, 2010

(54) Method for the Selective Enrichment of Double-Stranded Dna from Nucleic Acid Mixtures

(76) Inventors: Markus Müller, Dormagen (DE); Maria-Regina Kula, Munich (DE); Jürgen Hubbuch, Karlsruhe (DE); Andreas Frerix, Kalkar-Kehrum (DE)

Correspondence Address: Leon R Yankwich;Yankwich & Associates, 201 Broadway, Cambridge, MA, 02139, US

(21) Appl. No.: 11/991,947

(22) PCT Filed: Sep. 13, 2006
(86) PCT No.: PCT/EP2006/066335

§ 371 Date: Sep. 15, 2009

(30) Foreign Application Priority Data
Sep. 13, 2005 (EP) 05019914.0

(51) Int. Cl. C12N 15/00; C07H 1/00

(52) U.S. Cl. 435/320.1; 536/25.3

(57) ABSTRACT

The invention relates to a method for stripping undesired nucleic acid components from double-stranded DNA, in particular, super-coiled plasmid DNA. The method according to the invention is characterised by the steps: (a) provision of a mixture containing completely and/or partly double-stranded nucleic acids and optionally single-stranded nucleic acids; (b) resuspension of the mixture from step (a) in an aqueous, low-molarity buffer system with low ionic strength and low buffer effect; (c) adjusting conditions in the mixture from step (b), under which the completely and/or partly double-stranded nucleic acids are denatured; (d) further addition of buffer and a polymer component to the mixture from step (c); (e) incubation of the mixture from step (d) for a time which is sufficient for the formation of an aqueous two-phase system with an upper and lower phase; and (f) removal of the upper phase containing the single-strand nucleic acid and collection of the double-strand nucleic acid from the lower phase.

The present invention relates to a method for the selective enrichment of double-stranded DNA, in particular of supercoil plasmid DNA, from nucleic acid mixtures. The present invention is concerned with the stripping (removal) of single-stranded nucleic acids, such as for example ribonucleic acid (RNA), denatured genomic deoxyribonucleic acid (DNA) and/or partly denatured open circle plasmid DNA, from preparations containing double-stranded nucleic acids such as supercoil (sc) plasmid DNA.

In the prior part, there are numerous methods for isolating plasmid DNA for therapeutic applications, both on a kit and on a “high-throughput” (HT) scale, as well as on a production scale, which however in the majority of cases do not deal with a separation of genomic DNA (gDNA) (which under normal conditions is present in a double-stranded form) and/or open circle plasmid DNA (oc pDNA) from sc plasmid DNA (sc pDNA).

However, there are a series of publications, which deal specifically with this subject. In analytical applications, chromatographic methods are mainly to be found for the oc/sc separation of pDNA (e.g. TSKgel, DEAE-NPR and other products of the TSKgel series from Tosoh Biosciences), but capillary gel electrophoretic methods and a range of molecular biological techniques relating to sequence-specific hybridisation (e.g. so-called triple helix, etc.) are also used.

Some chromatographic technologies (PlasmidSelect resin from GE Healthcare and other HIC resins), which however are characterised by complex handling, high investment costs and above all by low yield efficiencies, are described for preparative processing of pDNA on a pilot and a production scale.

Recently, there have also been publications relating to the use of two-phase separations for the preparative isolation of plasmid DNA. In the works mentioned below, however, the problem of separating single-stranded DNA from double-stranded DNA has not been described at all or not technically satisfactorily, cf. for example Kepka C, Rhodin J, Lemmens R, Tjerneld F, Gustavsson P-E., 2004, Extraction of plasmid DNA from Escherichia coli cell lysate in a thermoseparating aqueous two-phase system*1, Journal of Chromatography A 1024(1-2): 95-104.; Frerix, A., Müller, M., Kula. M.-R., and Hubbuch J. Scalable recovery of plasmid DNA based on aqueous two-phase separation, Biotechnol. Appl. Biochem. (2005) 042, 57-66; Ribeiro S. C., Monteiro G. A., Cabral J. M. S., Prazeres D. M. F., 2002, Isolation of plasmid DNA from cell lysates by aqueous two-phase systems, Biotechnology and Bioengineering 78(4): 376-384.

For example, the described methods for oc/sc separation such as hydrophobic interaction chromatography (HIC) and thiophilic chromatography (Lemmens R., Olsson U., Nyhammar T., Stadler J., 2003, Supercoiled plasmid DNA: selective purification by thiophilic/aromatic adsorption, Journal of Chromatography B-Analytical Technologies in the Biomedical and Life Sciences 784(2): 291-300) and counter-current chromatography (Kendall D., Booth A. J., Levy M. S., Lye G. J., 2001, Separation of supercoiled and open-circular plasmid DNA by liquid-liquid counter-current chromatography, Biotechnology Letters 23(8): 613-619) are very time-consuming and very expensive due to low capacities and/or lack of yield, which must be seen as a disadvantage of these methods.

The object of the present invention is therefore to specify a method in which it is made possible to separate selectively (partially) denatured gDNA and oc pDNA as well as other single-stranded nucleic acids from double-stranded nucleic acid(s) such as sc plasmid DNA, without having to accept the above-mentioned disadvantages of the known methods. In particular, the object is to be seen in enabling pDNA to be separated from other nucleic acids.

The invention achieves this object by means of the method specified in the independent Claim 1. Further advantageous embodiments of the method according to the invention can be seen from the dependent claims, the description, the examples and the drawing.

The invention relates to a method for the specific stripping of single-stranded nucleic acids, such as for example RNA, denatured genomic DNA and partly denatured open circle plasmid DNA, from preparations, which likewise contain double-stranded nucleic acids such as supercoil plasmid DNA. In a special case, for example, the present invention enables selective denaturing of gDNA and oc pDNA as well as their subsequent separation by extraction in a two-phase system. This therefore involves polishing right down to double-stranded nucleic acids, such as sc pDNA for example. The method is distinguished by the fact that portions of double-stranded nucleic acids, such as genomic DNA, loop-building RNA and native double-stranded oc-pDNA, can be completely or partially selectively transferred into individual strands by denaturing, and subsequently selectively separated from double-stranded nucleic acid, such as sc Plasmid-DNA, with high efficiency and capacity in an aqueous two-phase system. The denaturing step can preferably be induced by strongly alkaline conditions (e.g. addition of NaOH, KOH etc.) or heat incubation (e.g. heating to ≧70° C., in particular ≧80° C., depending on the GC content of the nucleic acid). In comparison with existing conventional (e.g. chromatographic) methods, the advantages of the invention presented lie particularly in the considerably lower costs, the significantly faster speed of execution and the ability to more easily automate the methodology.

The present invention relates to a method for the selective stripping of partially and completely denatured nucleic acids from double-stranded nucleic acids, in particular sc pDNA. The method is particularly suitable for the manufacture of sc pDNA preparations on a pilot and production scale, e.g. for the manufacture of sc plasmid DNA for human genetic vaccination or for gene therapeutic applications, but because of its simplicity is also suitable for use in manual kit and automated high-throughput (HT) applications, e.g. in diagnostics. The method according to the invention is particularly well suited for the selective stripping of single-stranded nucleic acids and open circle (oc) plasmid DNA from preparations containing supercoil plasmid DNA.

When cleaning plasmid molecules for clinical or diagnostic use, the process development is focused on the product quality to be achieved on the one hand and on the resulting preparation costs (cost-of-goods, COGs) in proportion to this on the other. In this connection, the objective with regard to the required purity of the target molecule (e.g. pDNA) can differ considerably. The desired and necessary degrees of purity in clinical applications are considerably higher by comparison than those required in most diagnostic applications, for example. However, in both fields, the objective of process optimisation is to reduce the costs of cleaning to a minimum in order to make commercial applications possible. This objective can only be achieved by the development of highly resolution cleaning methods, as these at the same time enable the number of necessary cleaning steps to be kept as low as possible.

This objective (i.e. a most possible efficient cleaning of plasmid DNA) becomes all the more difficult the more physico-chemically similar the components to be stripped out are to the target molecule. So-called open circle (oc) plasmid DNA essentially differs from supercoil (sc) plasmid DNA only by a strand break or several strand breaks in one strand or both strands of the double helical structure of the plasmid molecule, which consequently also leads to steric differences between the two topological forms. oc pDNA is produced from sc pDNA predominantly by enzymatic or mechanical nicking of the sc pDNA, which is mainly present in vivo. In doing so, an sc pDNA is produced if, before the closing of two individual strands to form one double strand, one of the two strands or both strands are twisted so that, after closing to form the double strand, loops (“supercoils”) are formed due to the resulting stresses.

The present invention for the first time enables the desired separation of nucleic acids to be achieved highly selectively as well as extremely easily, quickly and in particular cost effectively. In doing so, an almost quantitative separation of (partially) single-stranded DNA (e.g. denatured oc pDNA) from the double-stranded DNA (e.g. sc pDNA) is achieved after careful treatment to obtain single and double-stranded nucleic acids in a subsequent two-phase separation, such as is described in WO 2004/106516 A1. This is achieved by means of inexpensive additives, which can be disposed of without any problems, with at the same time the high specific capacity of the described invention.

The present invention therefore describes the specific complete or partial denaturing of double-stranded nucleic acids, for instance by the effect of alkaline pH values of 11 or higher, or by means of heat. Such denaturing has the consequence that single-stranded nucleic acids, such as DNA or RNA, follow different distribution coefficients in phase systems compared with double-stranded nucleic acids, such as DNA, due to modified dilution characteristics.

In contrast to oc pDNA, for example, sc pDNA also denatures during the denaturing phase, but re-natures completely back to the supercoil double helix structure due to the three-dimensional topology and the resulting steric stabilisation of the so-called supercoils, e.g. after neutralising or cooling. Compared with double-stranded DNA, single-stranded DNA and RNA have a more hydrophobic surface, which can be contributed to the presence of free bases. Due to the different re-naturing and denaturing characteristics and the resulting structural characteristics, hydrophobicity and charge densities of oc and sc pDNA for example, a highly selective separation of the two plasmid topoisomers can be achieved by extraction in aqueous two-phase systems.

In the present invention, this mechanism is intentionally used to considerably amplify the normally extremely small differences between the surface characteristics of sc pDNA and oc pDNA as well as gDNA by deliberate selective denaturing, as a result of which a later separation can be carried out highly efficiently.

For the present invention, a buffer is added in step (d). A potassium phosphate buffer is preferably used here. In this case, the buffer particularly preferably contains a mixture of K₂HPO₄ and KH₂PO₄. The buffers according to the invention are preferably used with a pH value in the range from pH 5.8 to pH 8.5, and particularly preferably with a pH value in the range from pH 6.5 to pH 8. For example, a mixture of the stock solution of 3.83 M K₂HPO₄ and 2.45 M KH₂PO₄ and a PEG 800 concentration of 75% w/w (resulting in a pH value of ca. 7) can be particularly preferably used in the method according to the invention. In this case, K₂HPO₄ and KH₂PO₄ are used, for example in a concentration of 5-30% (w/w) referred to the two-phase system, preferably in a total concentration of 10-25% (w/w), and particularly preferably in a total concentration of 20% (w/w). The potassium phosphate is usually added in a temperature range between ice-cooled and room temperature. Room temperature as defined by the present invention designates a temperature range of 18 to 25° C. Preferably, an ice-cooled phosphate buffer is used in the method according to the invention. Advantageously, incubation is not necessary after adding the potassium phosphate; a mixing, which is as complete and uniform as possible, of the solution after adding the buffer is the decisive factor. If incubation should be carried out, however, the incubation period is usually about 1 to 15 minutes. Preferably, as mentioned above, the preparation is agitated, for example shaken hard, stirred or similar, during and/or after adding the salt components.

The polymer component, which is used according to the invention, is preferably polyethylene glycol (PEG). The polyethylene glycol is preferably used with a molecular weight having an arithmetic mean of 600 to 1000 g/mol, more preferably having an arithmetic mean of 700-900 g/mol and particularly preferably having an arithmetic mean of 750-880 g/mol, as one of the two components of the two-phase system. The PEG used in the present invention preferably consists of a mixture of polyethylene glycol with an average molecular weight of 600 g/mol (PEG 600) and polyethylene glycol with an average molecular weight of 1000 g/mol (PEG 1000). Both polyethylene glycols are commercially available (e.g. Fluka, Buchs, Switzerland). In this case, the ready-to-use PEG mixture consists, for example, of 30-50% (w/w) PEG 600 and 50-70% (w/w) PEG 1000, preferably 33-45% (w/w) PEG 600 and 55-67% (w/w) PEG 1000, particularly preferably of 36-40% (w/w) PEG 600 and 60-64% (w/w) PEG 1000 and quite particularly preferably of 38% (w/w) PEG 600 and 62% (w/w) PEG 1000.

The concentration of the PEG in the aqueous two-phase system according to the invention is chosen so that two phases form together with the salt components, wherein however the PEG concentration at which the double-stranded DNA, e.g. plasmid DNA, changes from the lower phase, in which it can be found at lower concentrations, to the upper phase is not exceeded. Preferably, the PEG content in the overall mixture is at least 10% (w/w) and is limited in an upwards direction by the concentration of PEG at which the double-stranded DNA (e.g. plasmid DNA) changes from the lower phase, in which it can be found at lower concentrations, to the upper phase. After adding PEG, the solution should preferably have a temperature of about 10 to 50° C., particularly preferably a temperature of about 15 to 40° C. After the formation of the phases, which can take from several minutes to hours depending on the volume of the preparation, the double-stranded DNA (e.g. plasmid DNA) will be found in the saline lower phase. As an option, the formation of the phases can be accelerated by centrifuging the preparation, as a result of which, advantageously, the time required for the method according to the invention is further reduced. The conditions under which such a centrifugation step is carried out are familiar to the person skilled in the art.

Compared with solid adsorptive phases, aqueous two-phase systems have the advantage that they have a considerably higher capacity for the double-stranded DNA (such as plasmid DNA) to be cleaned, which in practice is only limited by the solubility in the phases. Furthermore, the method can be scaled almost at will due to the very simple equipment necessary. But an automation, as well as independently thereof a production on an industrial scale, for example for the production of >>2 g highly cleaned plasmid DNA per preparation, can only be achieved easily with the simplifications described here. Likewise, with the present invention, the double-stranded DNA (such as plasmid DNA) can advantageously be freed to a very large extent from RNA and denatured gDNA, which in many applications, particularly in clinical applications, is a to some extent regulatory requirement. Plasmid DNA, which is polished using the method according to the invention, particularly after a primary cleaning step (for example by means of QIAGEN resin, QIAGEN, Hilden, Germany), is within the approval specifications currently accepted in gene therapy or genetic vaccination. In this way, large quantities of highly pure plasmid DNA can advantageously be produced with very little outlay on equipment while using non-toxic substances and with comparatively low costs. In this regard, it should be mentioned that, in comparison with other isolation methods, such as for example CsCl density gradient centrifugation or phenol extraction, the substances used in the two-phase system according to the invention are ecologically harmless and can be completely and easily removed from the cleaned plasmid DNA.

In the method according to the invention, the lower phase, which is produced in step (e) and which contains the double-stranded DNA, is separated from the upper phase, which contains the undesired nucleic acids, whereupon the desired double-stranded DNA can be obtained in enriched form from the lower phase (step (f)). Although a high degree of stripping can be achieved with just a single phase separation, the efficiency of the method according to the invention can be further increased by repeating steps (d) to (f) once to several times or by carrying out the extractions using the counter-current principle. It is expedient, for example, to repeat method steps (d), (e) and (f) one to three times. For extremely highly enriched double-stranded nucleic acid(s), such as sc plasmid DNA, steps (d) to (f) can also be repeated more than three times until the required purity is achieved. The double-stranded DNA (e.g. plasmid DNA) is to be found in the lower phase in each case. Carrying out this optional step leads to a repeated cleaning of the double-stranded DNA (e.g. plasmid DNA) and therefore to a further stripping of contaminants, such as RNA for example, from the double-stranded DNA (e.g. plasmid DNA).

Subsequent to the method according to the invention, it is expedient to isolate the double-stranded DNA (such as sc plasmid DNA), which is to be found in the lower phase. The isolation and desalination of the (plasmid) DNA from the lower phase produced in step (e) can be carried out by ultrafiltration, diafiltration or gel filtration for example. However, for the purpose of the present invention, any other method known to the person skilled in the art can be used for isolation and/or desalination of the (plasmid) DNA from the lower phase.

The aqueous low-molarity buffer used in step (b) is preferably a weak buffer, which has only a low ionic strength. The molarity of the buffer used is preferably not more than 100 mM, more preferably not more than 50 mM, and in particular not more than 10 mM.

Examples of suitable aqueous low-molarity buffer systems are a Tris buffer, a Tris/EDTA buffer, phosphate-buffered saline solution (PBS) or a citrate buffer, as well as other buffer systems, which appear to be suitable to the person skilled in the art.

For creating denaturing conditions in step (c), the pH value of the solution can be increased to 11 or above, or the temperature can be increased to 70° C. or higher. The pH value can be increased in the usual way by adding a strong base such as NaOH or KOH. When increasing the temperature, it is of advantage if the chosen temperature lies between about 70 and 95° C., in particular about 80 and 95° C. The optimum temperature here depends on the GC content of the nucleic acids present.

It has been shown to be particularly advantageous when the method according to the invention is carried out subsequent to a preliminary cleaning or preliminary separation, wherein any known cleaning method can be used for the preliminary cleaning/preliminary separation. A particularly suitable preliminary cleaning/preliminary separation is an aqueous two-phase separation, for example, such as is known from WO 2004/106516 A1. With the appropriate procedure, a very high degree of cleaning can be achieved. For instance, in this way it is possible to increase the content of sc pDNA in a sample, referred to the existing total quantity of nucleic acid, to 90% and more overall, in particular to 95% and more and even to 99% and more. Another especially suitable method for the preliminary cleaning/preliminary separation is anion exchange chromatography in which comparable percentage sc pDNA contents can be achieved in a sample.

The present invention is explained in more detail below with reference to examples.

### EXAMPLE 1

This example concerns the denaturing of oc pDNA under alkaline conditions and stripping in the two-phase system.

A pre-cleaned and concentrated plasmid DNA preparation containing oc pDNA and sc pDNA as well as the further nucleic acids RNA and partially double-stranded gDNA is incubated at a strongly alkaline pH value (>11). Under these conditions, a denaturing (strand separation) of the gDNA double helix and the double-strand RNA (e.g. tRNAs) is achieved, which can only be reversed in a limiting region with intact supercoil DNA. After transferring the denaturing preparation to a buffered phase system, which is optimised for the separation of oc/sc pDNA topoisomers, an efficient and highly resolvent separation of the oc pDNA, gDNA and the partially double-stranded RNA from the sc pDNA target molecule takes place.

This was carried out as follows. 5 g NaOH (0.4 M NaOH) were added to 15 g of a plasmid-containing starting solution (36 μg/ml, determined by means of HPLC). The reaction preparation was mixed and incubated at room temperature for 5 min. 20 g of potassium phosphate buffer (50% w/w, pH 7.4) and 10 g PEG 800 (75% w/w) were then added. This composition was in turn mixed well. After mixing, a typical clouding of the preparation occurred. The settling of the upper and lower phase can be accelerated by centrifugation (e.g. 5 min at 2000 g). The separated lower phase contains the cleaned sc pDNA while denatured DNA (oc pDNA and gDNA) can be seen as a white “smear” in the phase boundary (interphase, between lower and upper phase).

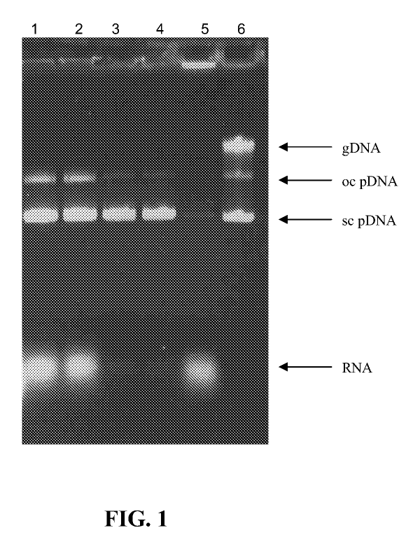

The results can be seen in the 0.8% agarose gel depicted in FIG. 1. The following can be seen in the figure:

| Lanes | Sample |
|---|---|
| 1 + 2 | Starting solution containing plasmid |
| 3 + 4 | Plasmid in desalinated lower phase following alkaline |
| | denaturing and two-phase extraction |
| 5 | Interphase from the aqueous two-phase separation, |
| | resuspended in TE (10 mM Tris/Cl, 1 mM EDTA, pH 8.0) |
| 6 | gDNA, pDNA standard |

Samples 1 and 2, and 3 and 4 were applied twice before and after cleaning in identical volumetric ratios. It can be clearly seen from the agarose gel shown in FIG. 1 that the proportion of oc pDNA (third band from the bottom) in the nucleic acids in traces 3 and 4, which have been subjected to a method according to the invention and which have been removed from the lower phase after aqueous two-phase separation, is greatly reduced compared with traces 1 and 2, which represent the starting solution. The sc pDNA target molecule (second band from the bottom) is present in a highly cleaned form. It can also be seen from traces 3 and 4 of FIG. 1 that the low molecular RNA residue (bottom band) of the sample has been practically quantitatively removed as a result of the treatment according to the invention of the nucleic acid sample. Finally, in trace 5 (interphase), only the low molecular RNA residues (bottom band) and denatured DNA can be seen in the form of pocket contamination.

### EXAMPLE 2

This example concerns the denaturing of oc pDNA by heat incubation and stripping in the two-phase system. A total of 350 μl containing pDNA (100 μg/ml) and gDNA (49 μg/ml) were prepared in TE. The preparation was then heated to temperatures of 70 to 95° C. (in 5° C. steps), incubated for 5 min in each case, and subsequently cooled on ice for 5 min in each case. 300 mg of the samples were then mixed with 300 mg TE, and 600 mg phosphate buffer (50%, w/w) and 300 mg PEG (75%, w/w) were added and mixed. Following this, the preparation was centrifuged. The total volume was 1.2 ml, of which 650 μl were accounted for by the lower phase, which contained the cleaned sc DNA.

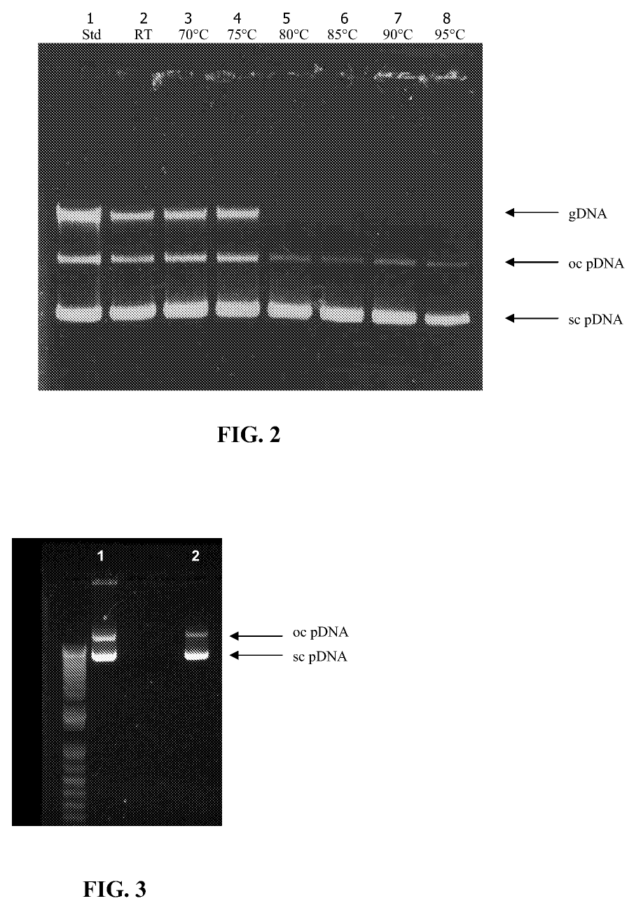

The results can be seen in the 0.8% agarose gel depicted in FIG. 2. The following can be seen in the figure:

| Lane | Sample |
|---|---|
| 1 | pDNA and gDNA at RT (standard (Std), corresponding to 100% yield) |
| 2 | pDNA and gDNA at RT, subsequently aqueous two-phase system |
| 3 | 5 min at 70° C.; subsequently on ice and aqueous two-phase system |
| 4 | 5 min at 75° C.; subsequently on ice and aqueous two-phase system |
| 5 | 5 min at 80° C.; subsequently on ice and aqueous two-phase system |
| 6 | 5 min at 85° C.; subsequently on ice and aqueous two-phase system |
| 7 | 5 min at 90° C.; subsequently on ice and aqueous two-phase system |
| 8 | 5 min at 95° C.; subsequently on ice and aqueous two-phase system |

The bottom band of the agarose gel shown in FIG. 2 shows sc pDNA. It can also be seen from the gel that, under the given conditions and for the plasmid pCMVP used, a denaturing temperature of 80° C. with 5 min incubation is sufficient to achieve a practically quantitative separation of gDNA and oc pDNA from sc pDNA.

### EXAMPLE 3

This example relates to the oc pDNA stripping (“polishing”) of a vaccination plasmid by means of alkaline denaturing and an aqueous two-phase system following primary cleaning by means of anion exchange chromatography.

The manufacture of plasmid DNA for gene therapeutic and genetic vaccinations is subject to stringent regulations and exacting specifications. Certain plasmid sequences tend to be “nicked” in preparation, i.e. to acquire single-strand breakages, and therefore to form high proportions of oc pDNA. The resulting proportions of oc pDNA must be stripped after initial cleaning. In the present case, this stripping was carried out by means of anion exchange chromatography.

The test was carried out as follows. 59.24 g of a plasmid solution (4 mg/ml) were added to 540.8 g of a Tris/EDTA buffer (pH value 8). 200 g NaOH (0.4 M) were then added and mixed. The mixture was then incubated for 5 minutes at room temperature. 700 g potassium phosphate buffer (50% w/w, pH 7.4) and 400 g PEG 800 (75% w/w, 60° C.) were subsequently added to the mixture. This was again mixed and centrifuged at 3000×g for 10 min. The lower phase (ca. 900 ml) contains cleaned sc pDNA, which is transferred to suitable formulation solutions by means of ultrafiltration or gel filtration for example.

The results can be seen in the 0.8% agarose gel depicted in FIG. 3. The following can be seen in the figure:

| | |
|---|---|
| Lane 1 (before “polishing”): | Total pDNA: 236.6 mg; |
| Pocket: | 4.1% mainly gDNA; |
| oc: | 28.4% (absolutely: 67.2 mg oc pDNA); |
| sc: | 67.5% (absolutely: 159.7 mg sc pDNA); |

| | |
|---|---|
| Lane 2 (after “polishing”) | Total pDNA: 168 mg |
| Pocket: | No detectable signals; |
| oc: | 14.9% (absolutely: 25 mg oc pDNA) |
| sc: | 85.1% (absolutely: 142 mg sc pDNA) |

As is shown by a comparison of the two traces 1 and 2 of the gel shown in FIG. 3, the oc pDNA band after the cleaning step (after “polishing”, see lane 2) is considerably weaker than before the cleaning step (before “polishing”, see lane 2). The quantitative results relating to this can be seen in the above table.

As can be seen from what is presented above, the quantity of oc plasmid pDNA in a biological sample can be reduced from more than 67 mg to 25 mg by means of the method according to the invention, i.e. a reduction of about 62% can be achieved. On the other hand, 142 mg of the 159.7 mg sc pDNA in the initial sample were recovered after carrying out the method according to the invention, which corresponds to a yield of about 89%.

What is claimed is:

1. A method for removing single-stranded nucleic acids from double-stranded nucleic acids, by comprising the following steps:
(a) Providing a mixture containing completely and/or partly double-stranded nucleic acids and optionally single-stranded nucleic acids;
(b) Resuspending the mixture from step (a) in an aqueous, low-molarity buffer system with low ion strength and low buffer effect;
(c) Adjusting conditions in the mixture from step (b), which lead to a reversible denaturing of a specified double-stranded nucleic acid or several specified double-stranded nucleic acids, wherein another nucleic acid or several other nucleic acids are irreversibly denatured;
(d) Adding a buffer and a polymer component to the mixture from step (c);
(e) Incubating the mixture from step (d) for a time which is sufficient for the formation of an aqueous two-phase system with an upper and a lower phase; and
(f) Removing the interphase and the upper phase containing the single-strand nucleic acid, and f collecting the double-strand nucleic acid from the lower phase.

2. The method according to claim 1, characterised in that the mixture from step (a) contains supercoil (sc) plasmid DNA.

3. The method according to claim 1, characterised in that the specified double-stranded nucleic acid from step (c), which can be reversibly denatured, is supercoil (sc) plasmid DNA.

4. The method according to claim 1, characterised in that the mixture from step (a) contains open circle (oc) plasmid DNA.

5. The method according to claim 1, characterised in that in step (f), single-stranded oc plasmid DNA in the upper phase is separated from double-stranded sc plasmid DNA in the lower phase.

6. The method according to claim 1, characterised in that the aqueous low-molarity buffer system according to step (b) with a low ionic strength and a low buffer effect has a molarity of up to 100 mM.

7. The method according to claim 1, characterised in that the aqueous low-molarity buffer system according to step (b) is selected from the group consisting of a Tris buffer, a Tris/EDTA buffer, a phosphate-buffered saline solution (PBS), and a citrate buffer.

8. The method according to claim 1, characterised in that the denaturing conditions according to step (c) are produced by increasing the pH value to 11 or higher with subsequent sufficient incubation.

9. The method according to claim 1, characterised in that the denaturing conditions according to step (c) are produced by increasing the temperature to 70° C. or higher and that immediate cooling takes place on completion of incubation.

10. The method according to claim 1, characterised in that the additional buffer added according to step (d) is a potassium phosphate buffer.

11. The method according to claim 1, characterised in that the polymer added according to step (d) is a polyethylene glycol (PEG), having a molecular weight of 600 to 1000 g/mol.

12. The method according to claim 1, characterised in that the enriched double-strand nucleic acid from the lower phase is further concentrated by ultrafiltration or gel filtration.

13. The method according to claim 12, characterised in that the enriched double-stranded nucleic acid in the lower phase is sc plasmid DNA.

14. The method according to claim 1, characterised in that steps (d), (e) and (f) are repeated at least once.

15. The method according to claim 14, characterised in that steps (d), (e) and (f) are repeated three times.

16. The method according to claim 1, characterised in that only the lower phase in step (e) contains sc plasmid DNA.

17. The method according to claim 1, characterised in that the method is carried out subsequent to a preliminary separation/preliminary cleaning.

18. The method according to claim 17, characterised in that the preliminary separation/preliminary cleaning is an aqueous nucleic acid two-phase separation or an anion exchange chromatography.

19. The method according to claim 11, characterised in that the polymer added according to step (d) is a polyethylene glycol (PEG), having a molecular weight of 700 to 900 g/mol.

20. The method according to claim 11, characterised in that the polymer added according to step (d) is a polyethylene glycol (PEG), having a molecular weight of 750 to 880 g/mol.
